(19) United States
(12) Patent Application Publication
(10) Pub. No.: US 2022/0089996 A1
(43) Pub. Date: Mar. 24, 2022

(54) BIOWASTE TREATMENT SYSTEM AND A METHOD OF BIOWASTE TREATMENT IN ASSOCIATION THEREWITH

(71) Applicant: E2S2 SYSTEMS PTE. LTD., Singapore (SG)

(72) Inventors: Yen Wah TONG, Singapore (SG); Jingxin ZHANG, Shanghai (CN)

(73) Assignee: E2S2 SYSTEMS PTE. LTD., Singapore (SG)

(21) Appl. No.: 17/423,282

(22) PCT Filed: Dec. 31, 2019
(86) PCT No.: PCT/SG2019/050651

§ 371 Date: Jul. 15, 2021

(30) Foreign Application Priority Data
Jan. 18, 2019 (SG) 10201900495U

(51) Int. Cl. C12M 1/00; C02F 11/04; B09B 3/65; B09B 3/40; C12M 1/36; C12M 1/02; C12M 1/34; C12P 5/02; C12M 1/33; C12M 1/107; C05F 17/979; C05F 17/60; C05F 17/70

(52) U.S. Cl. CPC C12M 29/00; C02F 2103/20; B09B 3/65; B09B 3/40; C12M 41/48; C12M 41/22; C12M 41/12; C12M 41/30; C12M 41/40; C12P 5/023; C12M 45/02; C12M 45/06; C12M 21/04; C12M 23/36; C12M 43/08; C05F 17/979; C05F 17/60; C05F 17/70; C02F 11/04

(57) ABSTRACT

A decentralized based biowaste treatment system for treating biowaste (i.e., organic matter type/based waste such as manure, sawdust and/or food scraps) by manner of anaerobic digestion (e.g., an anaerobic digestion based waste-to-resource system) and a method of biowaste treatment in association with the biowaste treatment system.

### CROSS REFERENCE TO RELATED APPLICATIONS

This application claims the priority to Singapore application No. 10201900495U, filed 18 Jan. 2019, the contents of which are incorporated herein by reference.

### FIELD OF INVENTION

The present disclosure relates generally to a method and system for treating biowaste (i.e., organic matter type/based waste such as manure, sawdust and/or food scraps) by manner of anaerobic digestion (e.g., an anaerobic digestion based system).

### BACKGROUND

Organic matter type waste can generally be generated in huge quantities. Examples of organic matter type waste (as will be referred to as “organic waste” hereinafter) can include food waste and/or horticulture waste.

For instance, based on the 2016 statistical report of National Environment Agency (of Singapore), Singapore produced around 791,000 tons of food waste that year, of which only 14% is recycled with the rest (i.e., non-recycled organic waste) awaiting disposal. Moreover, in year 2016, 320,500 tons of horticultural waste was generated.

Incineration based treatment systems (e.g., incineration plants) would conventionally be used for non-recycled organic waste. However, for such incineration based treatment systems, there are concerns as to pollution to the environment (e.g., air and water emissions from large scale incineration plants) in addition to concerns as to high capital and operating costs.

Moreover, the average moisture content of, for example, food waste can generally be estimated to be around 70%-85%. Appreciably, such moisture content can adversely impact the performance (e.g., incineration efficiency) of incineration based treatment systems.

To address such an issue (i.e., organic waste with moisture content), anaerobic digestion based treatment systems can be considered.

However, conventional treatment systems as discussed above generally adopt a centralized treatment approach which would require the collection and transportation of organic waste. This may adversely impact cost and//or efficiency of waste disposal and/or treatment.

The present disclosure contemplates that there is a need to improve the manner in which organic waste can be treated.

### SUMMARY OF THE INVENTION

In accordance with an aspect of the disclosure, there is provided a waste-to-resource system suitable for anaerobic digestion of biodegradable matter (i.e., biowaste) and resource generation (electricity, hot water and fertilizer), comprising: an anaerobic digester; a biogas power generator connected to the anaerobic digester; a circulating pump connected with and adapted to remove digestate from the anaerobic digester and reintroduce the digestate to the anaerobic digester to circulate the digestate; and a programmable controller for controlling the circulating pump, wherein the programmable controller is programed to activate the circulating pump to mix or circulate the digestate at intervals within the anaerobic digester.

In accordance with another aspect of the disclosure, there is provided a method of biowaste treatment in association with the aforementioned waste-to-resource system comprising: (a) feeding hydrolyzed biodegradable matter into an anaerobic digester; (b) switching between mixing and not mixing a digestate within the anaerobic digester at intervals; (d) removing biogas from the anaerobic digester; and (d) generating electricity with the biogas.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

Embodiments of the disclosure are described hereinafter with reference to the following drawings, in which:

FIG. 1 shows a waste-to-resource system which can include a pre-treatment subsystem portion, a treatment subsystem portion and a post-treatment subsystem portion, according to an embodiment of the disclosure;

FIG. 2 shows an exemplary implementation, which can include an anaerobic digestor, of the waste-to-resource system of FIG. 1, according to an embodiment of the disclosure;

FIG. 3 shows that the post-treatment subsystem can, in the context of the exemplary implementation of FIG. 2, include a biogas power generator and one or both of a heat exchanger part and a water tank part, according to an embodiment of the disclosure;

FIG. 4 shows an exemplary variation of the anaerobic digestor of FIG. 2, according to an embodiment of the disclosure; and

FIG. 5 shows a method of biowaste treatment in association with the waste-to-resource system of FIG. 1, according to an embodiment of the disclosure.

### DETAILED DESCRIPTION

The present disclosure generally relates to an anaerobic digestion based waste-to-resource type eco-system. Particularly, the present disclosure relates to a self-sustainable type waste-to-resource eco-system suitable for anaerobic digestion of biowaste.

More particularly, aspects of the present disclosure are directed to a waste-to-resource process for anaerobic digestion of biowaste to convert biodegradable organic waste (e.g., food waste, animal manure, organic matter/materials, biowaste, biomass, waste sludge, household biowaste, and/or the like) to, for example, produce/generate electricity, hot water and/or fertilizer. Specifically, in an exemplary application, the present disclosure contemplates the management of biodegradable matter and production of resources in terms of electricity, heat and/or fertilizer.

In accordance with an aspect of the disclosure, there is provided a waste-to-resource system, comprising: an anaerobic digester; a biogass power generator connected to the anaerobic digester; a circulating pump connected with and adapted to remove digestate from the anaerobic digester and reintroduce the digestate to the anaerobic digester to circulate the digestate; and a programmable controller for controlling the circulating pump, wherein the programmable controller is programed to activate the circulating pump to mix or circulate the digestate at intervals within the anaerobic digester.

Advantageously, having intermittent mixing as opposed to no mixing or continuous mixing, allows the anaerobic digestion process to happen more efficiently. A more efficient anaerobic digestion process allows the size of the anaerobic digester to be highly compact resulting in a portable decentralized waste to resource system that saves space and is easily controlled.

In various embodiment, the circulating pump connected with the anaerobic digester is for the purpose of intermittent mixing/circulation such that when the circulating pump is activated it mixes or circulates the contents in the anaerobic digester and when it is switched off or deactivated there is no mixing or circulation of the contents in the anaerobic digester. The circulating pump takes up a lot less space than a traditional stirrer such as a rotor blade stirrer. The use of a circulating pump also allows the size of the anaerobic digester to be highly compact resulting in a portable decentralized waste to resource system that saves space and is easily controlled.

In various embodiments the programmable controller may be activated at predetermined time intervals. In various embodiments the circulating pump may be programmed to mix or circulate the contents of the anaerobic digester at predetermined time intervals anywhere from 1 to 10 minutes (min) or any intermittent range there between. In various embodiments the circulating pump may be programmed to mix or circulate the contents of the anaerobic digester for 1 min, or 2 min, or 3 min, or 4 min, or 5 min, or 6 min, or 7 min, or 8 min, or 9 min, or 10 min. In various embodiments the circulating pump may be switched off or deactivated with no mixing or circulation for anywhere between 10 min to 59 min. In various embodiments the circulating pump may be switched off or deactivated with no mixing or circulation for 10 min, 20 min, 30 min, 40 min, 50 min or 60 min.

In various other embodiments the programmable controller may be programmed to mix or circulate the contents of the anaerobic digester at intervals determined by pH range, temperature range or pressure range. In such an embodiment when the pH, temperature or pressure reach a certain predetermined value or range the circulating pump is activated to mix or circulate the contents in the anaerobic digester and when the pH, temperature, or pressure is no longer in the predetermined value or range the circulating pump is switched off or deactivated resulting in no mixing or circulation of the contents in the anaerobic digester.

In various embodiments the anaerobic digester may be for producing biogas from the digestate, by subjecting the hydrolysed biodegradable matter to anaerobic digestion process (e.g., methanogenesis) when in use.

In various embodiments the anaerobic digester may be formed from a light weight material that is glass lined. In various embodiments the light weight material may be metal such as stainless steel or aluminum. In various embodiments the light weight material may be plastic. Glass is less reactive with the contents of the anaerobic digester in use but it needs to have an outer covering to block visual light into the system. Using a light weight material reduces the weight of the anaerobic digester allowing it to be more easily transported or a portable system.

In various embodiments the biogas power generator may be an engine generator (i.e., a biogas power generator) for producing electricity from biogas.

In various embodiments the waste-to-resource system may further comprises a storage tank, and a blender coupled to the waste storage tank, wherein the waste storage tank is connected to the anaerobic digester via an inlet thereof.

In various embodiments the blender is for converting biodegradable mater into small pieces, by subjecting the biodegradable matter to, for example, a grinding process. Such embodiments have the advantage that waste that has been blended, grinded, sliced into smaller pieces that present a greater surface area for digestion and allows the size of the anaerobic digester to be highly compact resulting in a portable decentralized waste to resource system that saves space.

In various embodiments the waste storage tank (i.e., a mixer) for storing and hydrolyzing biodegradable matter from the blended biodegradable matter, by subjecting the biodegradable matter to, for example, hydrolysis and/or acidogenesis.

In various embodiments the waste storage tank is connected to the anaerobic digester via a feeding pump for transferring hydrolyzed biodegradable matter to an anaerobic digester through the inlet.

In various embodiments the waste-to-resource system may further comprises one or more water jackets wrapped externally around the anaerobic digester. In various embodiments hot water may be run through the one or more water jackets to heat the anaerobic digester from about 50° C. to about 80° C., or between 50° C. and 80° C., or from about 50° C. to about 70° C., or from about 55° C. to about 65° C. In such embodiments the one or more water jackets may comprise one or more heating elements. This allows the one or more water jackets to heat the anaerobic digester to any one of the temperatures listed above.

In various embodiments the waste-to-resource system further comprises a solar heating system to heat the anaerobic digester. In various embodiments the solar heating system may be configured or adapted to heat the anaerobic digester. This allows the anaerobic digester to be heated directly to any one of the temperatures listed above. In various other embodiments the solar heating system may be configured to heat the anaerobic digester via one or more heating elements of the one or more water jackets. This allows the one or more water jackets to heat the anaerobic digester to any one of the temperatures listed above.

In various embodiments the waste-to-resource system may further comprises a biogas storage tank located between the anaerobic digester and the biogas power generator. In various embodiments a biogas pump may be used for transferring biogas into a biogas storage tank

In various embodiments the waste-to-resource system may further comprises one or more gas removal tank located between the anaerobic digester and the biogas storage tank.

In various embodiments the one or more gas removal tank may comprise a first removal tank (e.g., for removing H₂O from the biogas). The H₂O may be removed as steam. The steam may be condensed. The H₂O may be run through the one or more water jackets to heat the anaerobic digester as described herein above.

In various embodiments the one or more gas removal tank may comprise a second removal tank (e.g., for removing H₂S from the biogas).

In various embodiments the waste storage tank may further comprises a mixer. In various embodiments the mixer can include a mixing apparatus in an exemplary form of a plurality of laterally extending blades which can be rotatably driven by a motor.

In various embodiments the waste-to-resource system further comprises at least one sensor in the anaerobic digester for measuring acidity, temperature and/or pressure.

In various embodiments the waste-to-resource system further comprises a heat exchanger part to recover waste heat for, for example, producing hot water.

In various embodiments the waste-to-resource system, further comprising a safety valve carried by each of the anaerobic digester and the biogas storage tank.

In various embodiments the waste-to-resource system, further comprising a biogas compressor coupled to the biogas storage tank.

In various embodiment, the waste-to-resource system can, for example, include:
a waste sorting sub-system
a blender for converting biodegradable mater into small pieces, by subjecting the biodegradable matter to, for example, a grinding process
an organic waste storage tank (i.e., a mixer) for storing and hydrolyzing biodegradable matter from the blended biodegradable matter, by subjecting the biodegradable matter to, for example, hydrolysis and/or acidogenesis. The mixer can include a mixing apparatus in an exemplary form of a plurality of laterally extending blades which can be rotatably driven by a motor
a feeding pump for transferring hydrolyzed biodegradable matter to an anaerobic digester
an anaerobic digester for producing biogas from the hydrolyzed biodegradable matter, subjecting the biodegradable matter to anaerobic digestion process (e.g., methanogenesis). The anaerobic digestor can, for example, include one or more water jackets
a first removal tank (e.g., for removing H₂O from the biogas)
a second removal tank (e.g., for removing H₂S from the biogas)
a biogas pump for transferring biogas into a biogas storage tank
a biogas storage tank for storing biogas
an engine generator (i.e., a biogas power generator) for producing electricity from biogas
a heat exchanger part to recover waste heat for, for example, producing hot water
a solar heating system (i.e., an arrangement of solar panels) to heat the anaerobic digester to a mesophilic/thermophilic temperature. For example, the solar panel(s) can be connected to/with the water jacket of the anaerobic digester for the purpose of reactor heating

In one exemplary arrangement, the waste sorting machine, the blender, the organic waste storage tank, the feeding pump, the anaerobic digester, the first removal tank (i.e., H₂O remover), the second removal tank (i.e., H₂S remover), the biogas pump, the biogas storage tank, the engine generator, and the heat exchanger part can be successively positioned in order.

Moreover, in one embodiment, the waste-to-resource system can include one or both of at least one safety valve and at least one pressure meter. Specifically, at least one safety valve can be carried by each of the anaerobic digester and biogas storage tank. Furthermore, at least one pressure meter can be carried by each of the anaerobic digester and biogas storage tank.

In another embodiment, the waste-to-resource system can include a control panel which can be configured to control the operating process of the waste-to-resource system.

In yet another embodiment, the waste-to-resource system can include one or more sensors which can be carried by the anaerobic digester. The sensor(s) can, in combination with the control panel, be used for measuring acidity (i.e., pH), temperature and/or pressure.

In yet a further embodiment, the waste-to-resource system can include an electric meter which can be used for recording/measuring power. The electric meter can be coupled to the engine generator. In one example, the electric meter can be positioned after the engine generator.

According to another aspect of the disclosure, there is provided a method of biowaste treatment comprising: (a) feeding hydrolyzed biodegradable matter into an anaerobic digester; (b) switching between mixing and not mixing a digestate within the anaerobic digester at intervals; (d) removing biogas from the anaerobic digester; and (d) generating electricity with the biogas.

In various embodiments the method comprises grinding biodegradable matter; and hydrolyzing the same prior to feeding hydrolyzed biodegradable matter into the anaerobic digester.

In various embodiments the biodegradable matter is ground into smaller sizes such as 1 mm to 5 mm. Smaller pieces present a greater surface area for digestion and allows the size of the anaerobic digester to be highly compact resulting in a portable decentralized waste to resource system that saves space.

In various embodiments the hydrolyzing comprises mixing.

In various embodiments the method further comprises heating the anaerobic digester. In various embodiments heating the anaerobic digester may comprise heating the anaerobic digester from about 50° C. to about 80° C., or between 50° C. and 80° C., or from about 50° C. to about 70° C., or from about 55° C. to about 65° C. When anaerobic digestion is carried out from about 50° C. to about 80° C. it is more efficient. A more efficient anaerobic digestion process allows the size of the anaerobic digester to be highly compact resulting in a portable decentralized waste to resource system that saves space and is easily controlled.

In various embodiments the method further comprises storing the biogas. Where a larger volume of biodegradable matter moves through the system then it may be possible to run the generator continuously and there is no requirement for storage. However, in some embodiments when the volume of biodegradable matter used in the method is less than 50 kg per day it may be necessary to store the biogas to ensure there is enough biogas to generate electricity.

In various embodiments the method further comprises purifying the biogas. In various embodiments purifying the biogas comprises removing one or more impurities, such as H₂S and moisture, from the biogas to produce purified biogas. In one embodiment, a biogas storage tank may be provided for storing the purified biogas.

In various embodiments the intervals are predetermined time based intervals. In various embodiments a circulating pump may be programed to activate at predetermined time intervals. In various embodiments the circulating pump may be programmed to mix or circulate the contents of the anaerobic digester at the predetermined time based intervals anywhere from 1 to 10 minutes (min) or any intermittent range therebetween. In various embodiments the predetermined time based intervals for mixing is 1 min, or 2 min, or 3 min, or 4 min, or 5 min, or 6 min, or 7 min, or 8 min, or 9 min, or 10 min. In various embodiments the predetermined time based intervals for no mixing or circulation is anywhere between 10 min to 59 min. In various embodiments the predetermined time based intervals for no mixing or circulation is 10 min, 20 min, 30 min, 40 min, 50 min or 60 min.

In various embodiments the interval is calculated based on a sensor range wherein when a value calculated from the sensor reach a predetermined range a circulating pump is programmed to activate to mix the digestate within the anaerobic digester and when the value calculated from the sensor are outside the predetermined range the circulating pump is programmed to not mix or deactivate.

Generally, the present disclosure contemplates a waste-to-resource system (i.e., suitable for anaerobic digestion of biodegradable matter) which can include any one or any combination of the following subsystems:
a waste sorting subsystem
a pretreatment subsystem for producing blended waste from the biodegradable matter
an anaerobic digestion subsystem for producing biogas from the blended biodegradable matter
a biogas purification subsystem for removing one or more impurities, such as H₂S and moisture, from the biogas to produce purified biogas. In one embodiment, a biogas storage tank can be provided for storing the purified biogas
an engine power generation subsystem for producing, for example, electricity
a heat recovery subsystem to recover heat from the hot exhaust gas
a fertilizer generation sub-system
a solar heating sub-system for reactor heating

Preferably, the waste-to-resource system can be an integrated type anaerobic digestion waste-to-resource system.

The present disclosure contemplates that an integrated type anaerobic digestion waste-to-resource system can be a highly compact decentralized waste-to-resource system which can be useful for in-situ biowaste treatment. This can facilitate cost savings and/or efficiency, since waste transport and processing can be minimized and/or streamlined. Moreover, it is contemplated that a highly compact decentralized waste-to-resource system can be generally associated with a small footprint, thus saving space and creating an easily controlled anaerobic environment for the enrichment of anaerobic microorganisms.

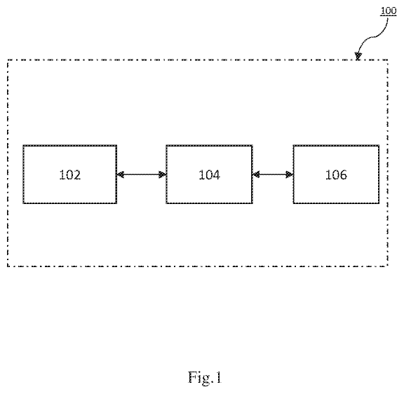

The foregoing will be discussed in further detail with reference to FIG. 1 to FIG. 5 hereinafter.

Referring to FIG. 1, there is shown a waste-to-resource system 100, according to an embodiment of the disclosure.

The waste-to-resource system 100 can include a pre-treatment subsystem portion 102, a treatment subsystem portion 104 and a post-treatment subsystem portion 106.

The pre-treatment subsystem portion 102 can be coupled to the treatment subsystem portion 104. The treatment subsystem portion 104 can be coupled to the post-treatment subsystem portion 106. Moreover, the treatment subsystem portion 104 can be associated with one or both of the aforementioned anaerobic digestion subsystem and biogas purification subsystem. In one embodiment, the treatment subsystem portion 104 can be further associated with the aforementioned solar heating sub-system. Additionally, the post-treatment subsystem portion 106 can be associated with any one of the aforementioned engine power generation subsystem, heat recovery subsystem, a fertilizer generation sub-system, or any combination thereof.

The pre-treatment subsystem portion 102 can be configured to receive biowaste (e.g., via a feeder 20), from a waste sorting subsystem (not shown) which can be configured to separate biowaste from general waste, and perform pre-treatment process on the received biowaste to produce pre-treated biowaste.

The pre-treatment process can, in one example, include grinding the received biowaste so that the received biowaste can be crushed to produce crushed biowaste. Specifically, received biowaste can be crushed so as to be dimensionally smaller in size as compared to when initially received (i.e., the size of biowaste when initially received can be dimensionally larger as compared to the size of the crushed biowaste).

The pre-treatment process can, in another example, include mixing the crushed biowaste in a manner so as to homogenize biodegradable matter. Specifically, crushed biowaste can, for example, be further treated in a manner so as to produce homogenized biowaste.

In this regard, the pre-treatment process can, for example, include one or both of a grinding process (i.e., to produce crushed biowaste) and a mixing process (i.e., to produce homogenized biowaste).

Appreciably, the pre-treatment subsystem portion 102 can be configured to perform, for example, one or both of a grinding process (i.e., to produce crushed biowaste) and a mixing process (i.e., to produce homogenized biowaste). In one embodiment, received biowaste can be treated via a grinding process to produce crushed biowaste and crushed biowaste can be treated via a mixing process to produce homogenized biowaste.

Further appreciably, pre-treated biowaste can, for example, include one or both of crushed biowaste and homogenized biowaste.

Pre-treated biowaste can be communicated from the pre-treatment subsystem portion 102 to the treatment subsystem portion 104 for further treatment.

The treatment subsystem portion 104 can, in one embodiment, be configured to receive and further process pre-treated biowaste in a manner so as to produce one or more products. Specifically, the treatment subsystem portion 104 can be configured to perform treatment process on the received pre-treated biowaste to produce one or more product(s).

The treatment process can, in one example, include anaerobic digestion (e.g., wet anaerobic digestion) of the received pre-treated biowaste to produce biogas. The treatment process can, in another example, include purifying the produced biogas to produce purified biogas.

In this regard, the treatment process can include one or both of an anaerobic digestion process (i.e., to produce biogas) and a purification process (i.e., to produce purified biogas).

In one example, the treatment subsystem portion 104 can include an anaerobic digestion subsystem (not shown) which can be configured to perform an anaerobic digestion process and/or a biogas purification subsystem (not shown) which can be configured to perform a purification process.

Appreciably, the treatment subsystem portion 104 can be configured to perform one or both of an anaerobic digestion process (i.e., to produce biogas) and a purification process (i.e., to produce purified biogas). In one embodiment, received pre-treated biowaste can be further processed via an anaerobic digestion process to produce biogas and the produced biogas can be further processed via a purification process to produce purified biogas.

Further appreciably, the product(s) produced can include one or both of biogas and purified biogas. The product(s) can be further communicated to the post-treatment subsystem portion 106. As an option, as will be discussed later in further detail, the product(s) can be stored prior to communication to the post-treatment subsystem portion 106.

The post-treatment subsystem portion 106 can be configured to receive and convert the product(s) (i.e., biogas and/or purified biogas) into a usable energy source (e.g., electricity).

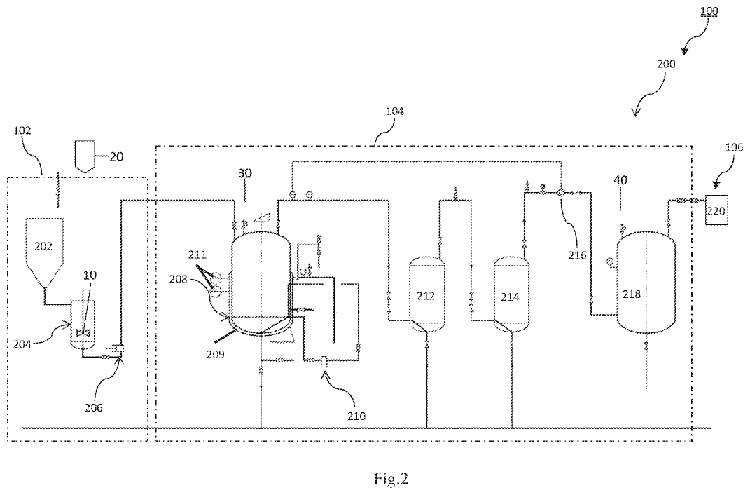

The foregoing will now be discussed in further detail, in the context of an exemplary implementation, with reference to FIG. 2 hereinafter.

FIG. 2 shows an exemplary implementation 200 of the waste-to-resource-system 100, according to an embodiment of the disclosure.

In the exemplary implementation 200, the pre-treatment subsystem portion 102 can include a blender 202 and a mixer 204. The pre-treatment subsystem portion 102 can further include a feeding pump 206. The blender 202 can be coupled to the mixer 204. The mixer 204 can be coupled to the feeding pump 206.

Moreover, in the exemplary implementation 200, the treatment subsystem portion 104 can include an anaerobic digester 208, a circulating pump 210, a first removal tank 212 and a second removal tank 214. The treatment subsystem portion 104 can further include a biogas compressor 216 and a biogas storage tank 218. The anaerobic digester 208 can be coupled to the circulating pump 210, controlled by a programmable controller (not shown). The anaerobic digester 208 can be coupled to the first removal tank 212 via piping and/or a pump. The first removal tank 212 can be coupled to the second removal tank 214. The second removal tank 214 can be coupled to the biogas compressor 216. The biogas compressor 216 can be coupled to the biogas storage tank 218.

Furthermore, in the exemplary implementation 200, the post-treatment subsystem portion 106 can include a biogas power generator 220. The post-treatment subsystem portion 106 will be discussed later in further detail with reference to FIG. 3. Generally, the feeding pump 206 can be coupled to the anaerobic digester 208 and the biogas storage tank 218 can be coupled to the biogas power generator 220. Therefore, the pre-treatment subsystem portion 102 can be coupled to the treatment subsystem portion 104 (i.e., via the feeding pump 206) and the treatment subsystem portion 104 can be coupled to the post-treatment subsystem portion 106 (i.e., via the biogas storage tank 218) as discussed earlier with reference to FIG. 1.

The exemplary implementation 200 will now be discussed generally, in turn, in further detail in the context of an exemplary operation and an exemplary arrangement hereinafter.

Regarding the exemplary operation, biowaste can be fed to the blender 202 (e.g., via a feeder 20) for processing by manner of a grinding process. After completion of the grinding process (i.e., to produce crushed biowaste) in the blender 202, the biodegradable matter (i.e., crushed biowaste) in the blender 202 can be transferred (i.e., communicated) to the mixer 204 for one or both of mixing and storage (e.g., the mixer 204 can be in the form of a combination of a mixing apparatus 10 and an organic waste storage tank where the mixing apparatus 10 can be disposed within the organic waste storage tank). After subsequent completion of the mixing process (i.e., to produce homogenized biowaste) by the mixer 204, the biodegradable matter (i.e., homogenized biowaste) carried by the mixer 204 can be transferred (i.e., communicated) to the anaerobic digester 208, via the feeding pump 206, for performing an anaerobic digestion process (i.e., for producing biogas). During the anaerobic digestion process, the circulating pump 210, controlled by a programmable controller (not shown), can be used to perform the function of circulation/mixing the digestate within the anaerobic digester 208.

Moreover, in one embodiment, digestate can be produced during the anaerobic digestion process and can be communicated from the waste-to-resource system 100 via the circulating pump 210. The present disclosure contemplates that such digestate can be used as fertilizer. Specifically, the present disclosure contemplates that such digestate (i.e., communicated from circulating pump 210) can be used as fertilizer after post-treatment. Generally, post-treatment can include processes such as dewatering, heating and/or drying. For example, the aforementioned digestate can be dewatered by a centrifuge separator (not shown) to form liquid fertilizer and solid fertilizer. Waste heat (i.e., a by-product) from, for example, the biogas power generator 220 can be used to heat liquid digestate to kill bacterial pathogens before it is used as liquid fertilizer. The solid fertilizer can, for example, be dried by the waste heat from, for example, the biogas power generator 220 to form/produce powdered fertilizer.

After subsequent completion of the anaerobic digestion process in the anaerobic digester 208, the biogas in the anaerobic digester 208 can be transferred (i.e., communicated) to the first removal tank 212 (e.g., for the removal of H₂O) and the second removal tank 214 (e.g., for the removal of H₂S) to perform the purification process so as to produce purified biogas (i.e., removal of H₂S and H₂O components in the biogas). After completion of the purification process, the purified biogas can be transferred (i.e., communicated) to the biogas storage tank 218, via the biogas compressor 216, for storage. Purified biogas can be transferred (i.e., communicated) from the biogas storage tank 218 to the biogas power generator 220 for generating electricity.

Moreover, regarding the exemplary operation, in one embodiment, during feeding or loading of biowaste to the waste-to-resource system 100, the blender 202 can be open to the feeder 20 (e.g., a source from which biowaste can be fed to the blender 202). The blender 202 can be connected through a suitable opening, conduit or piping mechanism to the mixer 204, wherein the opening can be configured for emptying the processed biowaste from the blender 202 to the mixer 204. Specifically, after the grinding process at the blender 202, the blended/crushed/ground biowaste in the blender 202 can be transferred to the mixer 204 via the opening of a valve. The opening may be activated (i.e., opened or closed) manually or automatically.

Additionally, regarding the exemplary operation, the mixer 204 can be configured to operate for several minutes before the feeding pump 206 is configured to transfer pre-treated biowaste to the anaerobic digester 208. The present disclosure contemplates that doing so can provide an advantage of homogeneous feeding and/or a further advantage of preventing overload (i.e., of biowaste) as solid biowaste can settle down at the bottom of the mixer 204.

Furthermore, regarding the exemplary operation, the mixer 204 can be connected through a suitable piping to the feeding pump 206, wherein the opening of such a suitable piping can be controlled for emptying the pre-treated biowaste from the mixer 204 to the anaerobic digester 208. The opening of such suitable piping may be activated (i.e., opened or closed) automatically or manually, using a general control panel known to a person having ordinary skill in the computer arts. An example of such a mechanism is an electric relay located in the control panel. The electric relay, when activated, allows for the start-up of the feeding pump 206 to transfer the pre-treated biowaste from mixer 204 to the anaerobic digester 208.

Moreover, regarding the exemplary operation, the anaerobic digester 208 can generally be configured for performing the process of anaerobic digestion for biogas production. More particularly, the anaerobic digester 208 can be configured for producing biogas from pre-treated biowaste (e.g., hydrolyzed biodegradable matter). The anaerobic digester 208 can, in one embodiment, includes a safety valve (e.g., safety valve 30 located on the top of the anaerobic digester 208) for reducing the risk of the anaerobic digester 208 from explosion as biogas is produced inside the anaerobic digester 208. More particularly, the safety valve 30 can be useful as a safety mechanism (i.e., for preventing build-up of excessive pressure within the anaerobic digester 208 in the event of, for example, access of biogas out of the anaerobic digester 208 to the first removal tank 212 being unintentionally blocked).

Yet furthermore, regarding the exemplary operation, the biogas compressor 216 can be used for transferring purified biogas to the biogas storage tank 218. More particularly, the biogas compressor 216 can be configured to begin transfer of purified biogas to the biogas storage tank 218 when the pressure within one or more of the preceding parts/elements (e.g., within the anaerobic digester 208) reach a certain/predetermined level. Conversely, when pressure (e.g., within the anaerobic digester 208) drops to a certain/predetermined level, the biogas compressor 216 can be configured to stop transfer of purified biogas to the biogas storage tank 218. In one embodiment, the biogas storage tank 218 can include a safety valve (e.g., safety valve 40 located on the top of the biogas storage tank 218) for reducing the risk of tank explosion as purified biogas can be stored within the biogas storage tank 218. More particularly, the safety valve 40 can be useful as a safety mechanism to, for example, prevent excessive build-up of pressure within the biogas storage tank 218.

Additionally, regarding the exemplary operation, the biogas power generator 220 can be configured to convert purified biogas, communicated from the biogas storage tank 218, to electricity. For example, after the biogas storage tank 218 has reached maximum storage capacity (i.e., of the purified biogas), the purified biogas stored in the biogas storage tank 218 can be communicated to the biogas power generator 220 via a safety valve and a flow rate controlling valve for generating electricity. The biogas power generator 220 can include an opening which can be activated (i.e., opened or closed) manually or automatically.

The above discusses the exemplary implementation 200 generally in the context of an exemplary operation.

The exemplary implementation will now be generally discussed in further detail in the context of an exemplary arrangement hereinafter.

Regarding the exemplary arrangement, the blender 202, the mixer 204, the feeding pump 206, the anaerobic digester 208, the circulating pump 210, the first removal tank 212, the second removal tank 214, the biogas compressor 216, the biogas storage tank 218 and the biogas power generator 220 can be, successively, carried by (e.g., disposed within) a twenty feet container. Specifically, the blender 202 can be positioned before the mixer 204, the mixer 204 can be positioned before the feeding pump 206, the feeding pump 206 can be positioned before the anaerobic digester 208, the circulating pump 210 can be positioned adjacent/at the bottom of the anaerobic digester 208, the anaerobic digester 208 can be positioned before the first removal tank 212, the first removal tank 212 can be positioned before the second removal tank 214, the second removal tank 214 can be positioned before the biogas compressor 216, the biogas compressor 216 can be positioned before the biogas storage tank 218, the biogas storage tank 218 can be positioned before the biogas power generator 220.

Earlier mentioned, the post-treatment subsystem portion 106 can include a biogas power generator 220, according to an embodiment of the disclosure. The present disclosure contemplates that the post-treatment subsystem portion 106 can further include, as shown in FIG. 3, one or both of a heat exchanger part 302 and a water tank part 304, according to an embodiment of the disclosure.

As shown, the biogas power generator 220 can be coupled to the heat exchanger part 302 and the heat exchanger part 302 can be coupled to the water tank part 304.

The heat exchanger part 302 can be configured to recover waste heat associated with exhaust emissions. Moreover, the water tank part 304 can carry cold water which can be communicated (e.g., pumped) to the heat exchanger part 302 so as to absorb heat from the exhaust emission. Communicated water can then be heated and stored in the water tank part 304.

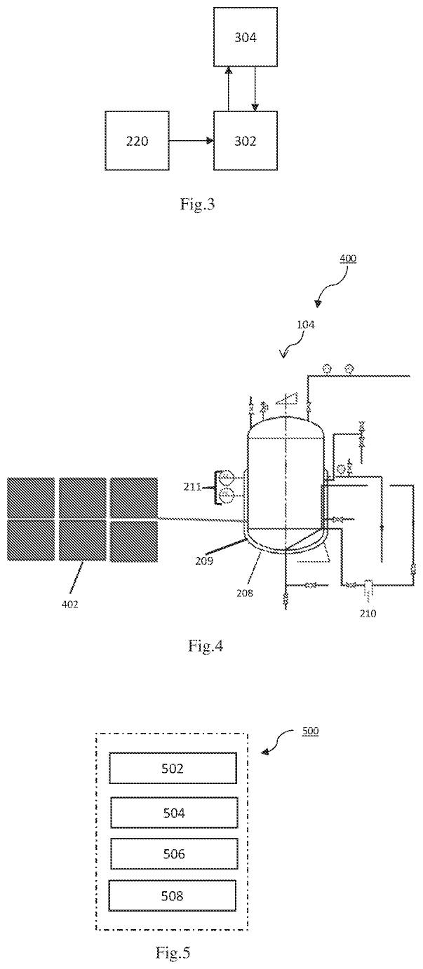

FIG. 4 shows an exemplary variation 400 of the anaerobic digester 208 of FIG. 2, according to an embodiment of the disclosure.

Regarding the exemplary variation 400, the anaerobic digester 208 can include an arrangement of solar panels 402. Specifically, the anaerobic digester 208 can be configured to carry an arrangement of solar panels 402.

The arrangement of solar panels 402 can be useful for facilitating self-heating. The arrangement of solar panels 402 can be also useful for improving organic loading rate and/or methane yield.

The present disclosure contemplates that the treatment subsystem portion 104 can, in one embodiment, be considered to be a self-heating subsystem (i.e., a self-sustainable subsystem) in view of the arrangement of solar panels 402. In this regard, the arrangement of solar panels 402 can be considered to be associated with the aforementioned solar heating sub-system.

For example, the anaerobic digester 208 can include one or more water jackets 209 and the arrangement of solar panels 402 can be configured to heat the water jacket(s). In this regard, water carried by the water jacket(s) can, for example, be heated to produce hot water.

In one embodiment, the arrangement of solar panels 402 can be carried atop a portion of the treatment subsystem portion 104 (e.g., atop the anaerobic digester 208) and a water jacket 209 can be wrapped externally around the digester 208. The hot water can heat up the anaerobic digester 208 to about 55-60° C.—this can facilitate an increase in activity of certain microbes in the anaerobic digester 208, thus allowing the microbes to convert biowaste (e.g., food wastes) into biogas at a comparatively faster rate (i.e., as compared to when the anaerobic digester 208 is not heated by the hot water).

For example, the arrangement of solar panels 402 can be connected to/with the water jacket 209 of the anaerobic digester 208 through a converter (not shown). There can be one or more heating elements (not shown) (e.g., four heating bars) carried by (e.g., inside) the water jacket. More specifically, the arrangement of solar panels 402 can be configured to generate direct current based power (i.e., DC power) which can in turn be converted to alternating current based power (i.e., AC power). AC power can be used to heat the heating element(s) (e.g., carried within the water jacket 209) for the purpose of reactor heating.

In yet another embodiment, the waste-to-resource system 100 can include one or more sensors 211 carried by the anaerobic digester 208. The sensor(s) 211 can, in combination with the control panel (not shown), be used for measuring acidity (i.e., pH), temperature and/or pressure. In yet another embodiment, the sensor(s) 211 can, in combination with the programmable controller (not shown), be used for activating or deactivating the circulating pump 210.

In view of the foregoing discussion, it is appreciable that anaerobic digestion can generally be considered to relate to a collection of processes by which microorganisms breakdown biodegradable materials (i.e., biowaste) in the absence of oxygen. During anaerobic digestion, biodegradable matter can be decomposed by anaerobic bacteria to produce a gaseous by-product (i.e., biogas). Biogas can generally include methane, carbon dioxide, and hydrogen sulphide. Anaerobic digestion can occur naturally but can also be used for industrial or domestic applications such as waste management and/or fuel production. In various industrial settings, anaerobic digestion can be used for treating biodegradable matter/materials, such as organic matter, biowaste, biomass, household biowaste, food waste, and/or the like, and to produce biogas as a result. As discussed earlier, anaerobic digestion can be conducted in devices known as anaerobic digesters (i.e., the anaerobic digester 208 of FIG. 2), which can treat/process biodegradable matter (i.e., biowaste) in the presence of anaerobic bacteria and heat, and in an oxygen-free environment.

The present disclosure contemplates that conventional centralized anaerobic digestion waste-to-resource processes generally do not facilitate space efficiency as the size associated with a conventional anaerobic digestion system would have to be increased in order to improve efficiency. Moreover, such conventional anaerobic digestion systems are generally not efficient (e.g., additional transportation of biowaste may be required and cost associated with such additional transportation).

Appreciably, the waste-to-resource system 100 of the present disclosure is a distributed and highly compact hybrid anaerobic digestion-based waste-to-resource system with the technologies of wet anaerobic digestion, high solids anaerobic digestion, biogas purification and IC engine power. The self-heating feature (i.e., arrangement of solar panels 402) can potentially enhance the efficiency of the anaerobic digestion processes of the biodegradable matter, such as food waste with high solid content, thereby improving the biogas yield and saving energy. There is thus a high biogas potential from the treatment or processing of the biodegradable matter, i.e. there is greater amount of useful materials, e.g. biogas, generated and reduced waste sludge or residual waste left over at the end of the anaerobic process. Furthermore, the present disclosure contemplates that units/parts/elements (e.g., the high efficient anaerobic digester 208 and/or other units such as the first removal tank 212) can be integrated within, for example, a container in a manner such that the overall space (and volume) required by the waste-to-resource system 100 system can be reduced. Therefore, it is appreciable that the waste-to-resource system 100 can have a smaller footprint so that it can potentially be used in location(s)/place(s) with limited space.

In this regard, the waste-to-resource system 100 can be decentralized in the sense that the waste-to-resource system 100 can be located where the wastes are being produced (i.e., the waste-to-resource system 100 can be located/implemented at the location of the waste source). This is in contrast to conventional centralized based systems where food wastes have to be collected (i.e., from the location such food wastes are produced) and brought to a centralized based system for treatment. Hence, the waste-to-resource system 100 can be utilized locally (i.e., at the location of production of biowaste). For example, the waste-to-resource system 100 can be located/implemented in food and beverage locations/establishments (e.g., hawker centers and/or restaurants etc.), thus mitigating the need for collection and transportation of food wastes for further treatment.

It would be appreciated that the waste-to-resource system 100 can be a distributed and highly compact hybrid anaerobic digestion-based waste-to-resource system with high efficiency and/or multi-production capabilities. Specifically, the waste-to-resource system 100 can relate to a combined compact system which can be capable of providing a promising solution for organic waste treatment. Moreover, to enhance the efficiency of the waste-to-resource system 100, co-digestion of different organic wastes can be conducted/performed. The waste-to-resource system 100 may also be associated with the potential to reduce waste transportation demands and costs associated therewith, and also capital investment requirement.

Performance of the waste-to-resource system 100 can be benchmarked based on an exemplary parameter. The exemplary parameter can, for example, be Methane production.

Based on an experiment conducted in the context of the exemplary implementation 200, biogas produced by the anaerobic digester 208 had on average a methane composition of 61%. Removal of almost 100% of H₂S can potentially be possible. CH₄ composition in the biogas storage tank 218 was maintained at above 60% during the course of the experiment.

Readings for the amount of gas produced were taken from Day 37 onwards when the waste-to-resource system 100 was fully operational. It could be observed that whenever load was increased, methane yield decreased during the first few loads. The specific methane yields then increased to and were maintained at around 0.55-0.60 L CH4/g VS. Methane yields for higher load (20 and 30 kg) were slightly lower than that of a lower load (10 kg); however, no significant decrease was observed, indicating that the reactor had not reached its maximum Organic Loading Rate (OLR). The average specific methane yields can be summarized as follows:

| Total Feed | Days of stable | OLR | Specific methane yield |
|---|---|---|---|
| (kg/d) | operation (d) | (g VS/L) | (L CH₄/g VS) |
| 10 | 4 | 1.8 | 0.64 ± 0.03 |
| 15 | 4 | 2.7 | 0.35 ± 0.03 |
| 20 | 4 | 3.6 | 0.56 ± 0.02 |
| 30 | 3 | 5.4 | 0.55 ± 0.01 |

Compared to known values, methane yield per the experiment of the present disclosure was relatively high. As can be noted above, the highest value is 0.73 L CH₄/g VS, with a single-stage reactor (i.e., anaerobic digester 208) while using fruit seeds (which are rich in protein) as the feed (i.e., biowaste fed to the blender 202). The present disclosure contemplates that the notable high methane yield could be attributed to feedstock compositions. For example, where food waste (i.e., biowaste) collected contained large amounts of meat and soy products (i.e., which are rich in protein that has high methane generation potential), 1-ton food waste can potentially produce 200-400 kwh electricity.

FIG. 5 shows a method of biowaste treatment 500 in association with the waste-to-resource system 100, according to an embodiment of the disclosure.

The method of biowaste treatment 500 can include a feeding step 502, a pre-treatment step 504, a treatment step 506 and a post-treatment step 508.

In regard to the feeding step 502, biowaste can be fed via a feeder (i.e., label 20 of FIG. 2) to the waste-to-resource system 100.

In regard to the pre-treatment step 504, biowaste can be processed/treated in the earlier discussed manner of grinding biodegradable matter in relation to the pre-treatment subsystem portion 102 to produce pre-treated biowaste.

In regard to the treatment step 506, pre-treated biowaste can be processed/treated in the earlier discussed manner of mixing intermittently and/or heating in relation to the treatment subsystem portion 104 to produce one or more products (e.g., biogas and/or purified biogas).

In regard to the post-treatment step 508, the product(s) can be converted into a usable energy source (e.g., electricity) in the earlier discussed manner of generating electricity in relation to the post-treatment subsystem portion 106.

It should be further appreciated by the person skilled in the art that variations and combinations of features described above, not being alternatives or substitutes, may be combined to form yet further embodiments.

In one example, biodegradable matter can include organic substrates of food waste or household waste that can be selected and ground into small particles. Therefore, it would be apparent to a person having ordinary skill in the art that the anaerobic digestion process can be applied not only to the anaerobic digestion of household biowaste, the anaerobic digestion process can further be applied to/in other facilities that produce organic waste matter.

In another example, the pre-treatment process can yet further include an additional process where inoculants can be added into biowaste (e.g., food waste) for implanting microorganisms or anaerobic bacteria therein.

In a yet another example, biowaste carried by the mixer 204 can be subjected to a chemical process of hydrolysis. Specifically, biowaste carried by the mixer 204 can be subjected to a chemical process of hydrolysis of the anaerobic digestion process. During the hydrolysis process, hydrolyzed biowaste (i.e., biodegradable matter) can be produced from biodegradable matter. More particularly, macromolecular organic matter in the biowaste (i.e., biodegradable matter) can be converted into smaller molecular substances and organic acids of the hydrolyzed biodegradable matter. The residence time of the biodegradable matter and the resultant hydrolyzed biodegradable matter in the mixer 204 can, for example, be up to 1 day. After the chemical process of hydrolysis performed at the mixer 204 of the anaerobic digestion process, the acidized biodegradable matter in the mixer 204 can be transferred to the anaerobic digester 208 via the feeding pump 206. It is generally contemplated that hydrolyzed biodegradable matter will become more soluble.

In yet a further example, it was earlier mentioned that the first removal tank 212 can, for example, be used for the removal of H₂O and the second removal tank 214 can, for example, be used for the removal of H₂S. It may be appreciated that the first removal tank 212 can, for example, be used for the removal of H₂S and the second removal tank 214 can, for example, be used for the removal of H₂O.

In an additional example, the waste-to-resource system 100 can include a programmable controller such as an automotive computer control system (not shown) which can be configured to perform smart data recording and communication (e.g., transmission). The present disclosure contemplates that the automotive computer control system can be useful for facilitating ease of operation of the waste-to-resource system 100 and/or for facilitating smart control in the event of variance of environment within, for example, the anaerobic digester 208.

In yet an additional example, the waste-to-resource system 100 can include an airtight control system (not shown) which can facilitate an odorless experience for the user(s) of the waste-to-resource system 100. For example, one or more portions of the post-treatment subsystem portion 106 may be associated with the airtight control system.

Moreover, in another additional example, the waste-to-resource system 100 can include one or both of at least one safety valve (i.e., labels 30 and 40 in FIG. 2) and at least one pressure meter. Specifically, at least one safety valve can be carried by each of the anaerobic digester and biogas storage tank. Furthermore, at least one pressure meter (not shown) can be carried by each of the anaerobic digester and biogas storage tank. Additionally, the mixer 204 can include a mixing apparatus (i.e., label 10 in FIG. 2) in an exemplary form of a plurality of laterally extending blades which can be rotatably driven by a motor (not shown).

In the foregoing manner, various embodiments of the disclosure are described for addressing at least one of the foregoing disadvantages. Such embodiments are intended to be encompassed by the following claims, and are not to be limited to specific forms or arrangements of parts so described and it will be apparent to one skilled in the art in view of this disclosure that numerous changes and/or modification can be made, which are also intended to be encompassed by the following claims.

1. A waste-to-resource system, comprising:
an anaerobic digester;
a biogas power generator connected to the anaerobic digester;
a circulating pump connected with and adapted to remove digestate from the anaerobic digester and reintroduce the digestate to the anaerobic digester to circulate the digestate; and
a programmable controller for controlling the circulating pump, wherein the programmable controller is programed to activate the circulating pump to mix or circulate the digestate at predetermined intervals within the anaerobic digester wherein the interval is a predetermined time based interval.

2. The waste-to-resource system according to claim 1, further comprising a waste storage tank, and a blender coupled to the waste storage tank, wherein the waste storage tank is connected to the anaerobic digester via an inlet thereof.

3. The waste-to-resource system according to claim 1, further comprising one or more water jackets wrapped externally around the anaerobic digester.

4. The waste-to-resource system according to claim 3, wherein the one or more water jackets comprise one or more heating elements.

5. The waste-to-resource system according to claim 1, further comprising a solar heating system adapted to heat the anaerobic digester.

6. The waste-to-resource system according to claim 1, further comprising a biogas storage tank located between the anaerobic digester and the biogas power generator.

7. The waste-to-resource system according to claim 6, further comprising one or more gas removal tank located between the anaerobic digester and the biogas storage tank.

8. The waste-to-resource system according to according to claim 2, wherein the waste storage tank further comprises a mixer.

9. The waste-to-resource system according to claim 1, further comprising at least one sensor in the anaerobic digester for measuring acidity, temperature and/or pressure.

10. The waste-to-resource system according to claim 6, further comprising a safety valve carried by each of the anaerobic digester and the biogas storage tank.

11. The waste-to-resource system according to claim 6, further comprising a biogas compressor coupled to the biogas storage tank.

12. A method of biowaste treatment comprising:
feeding hydrolyzed biodegradable matter into an anaerobic digester;
switching between mixing and not mixing a digestate within the anaerobic digester at intervals wherein the interval is a predetermined time based interval;
removing biogas from the anaerobic digester; and
generating electricity with the biogas.

13. The method according to claim 12, further comprising grinding biodegradable matter; and hydrolyzing the same prior to feeding hydrolyzed biodegradable matter into the anaerobic digester.

14. The method according to claim 12, further comprising heating the anaerobic digester.

15. The method according to claim 12, further comprising storing the biogas.

16. The method according to claim 12, further comprising purifying the biogas.

17. The method according to claim 12, further comprising compressing the biogas.

18. The method according to claim 12, wherein hydrolyzing further comprises mixing.

19.-20. (canceled)
